(19) United States
(12) Patent Application Publication
(10) Pub. No.: US 2005/0272106 A1
(43) Pub. Date: Dec. 8, 2005

(54) Methods and kits for detection of multiple pathogens

(76) Inventors: Norman Moore, Portland, ME (US); Cheryl Bailey, Windham, ME (US); Roger Piasio, Cumberland, ME (US)

Correspondence Address: T. D. FOSTER, 12760 HIGH BLUFF DRIVE, SUITE 300, SAN DIEGO, CA, 92130, US

(21) Appl. No.: 11/062,211

(22) Filed: Feb. 17, 2005

Related U.S. Application Data
(60) Provisional application No. 60/545,718, filed on Feb. 17, 2004.

(51) Int. Cl. G01N 33/554; G01N 33/569

(52) U.S. Cl. 435/7.32

(57) ABSTRACT

The present invention relates to methods of simultaneously detecting multiple infectious organisms, including methods of simultaneously determining the presence or absence in a sample of one or more members of a group of pathogens including at least one viral pathogen and at least one non-viral pathogen. The present invention also discloses and claims kits for carrying out the methods of the invention.

### BACKGROUND OF THE INVENTION

### FIELD OF THE INVENTION

The present invention relates generally to the field of medical diagnostics, and particularly to methods for simultaneously detecting multiple infectious organisms, including methods for simultaneously detecting viral and bacterial pathogens.

Infection of an individual by more than one pathogenic organism is a common phenomenon. Mixed viral infections (that is, concurrent infection by more than one type of virus) are not unusual (Waner (1994) Clin. Microbiol. Rev., 7:143-151). Mixed viral and bacterial infections have also been well documented, see, for example, Hietala et al (1989) Pediatr. Infect. Dis. J, 8:683-686, and Korppi (1999) Pediatr. Pulmonol. Suppl., 18: 110, which are incorporated by reference in their entirety herein.

Viral infections of the respiratory tract are among the most common human diseases, and include, but are not limited to, infection by respiratory syncytial virus (RSV) types A and B, influenza A, B, and C viruses, human parainfluenza viruses (HPIV) 1, 2, and 3, human metapneumovirus (hMPV), measles virus, adenoviruses (AV), rhinoviruses, coronaviruses, and enteroviruses. See, for example, Mackie (2003) Paediatr. Respir. Rev., 4:84-90; and Kahn (2003) Curr. Opin. Infect. Dis., 16:255-258, which are incorporated by reference in their entirety herein. Immunocompromised patients are vulnerable to respiratory infections by additional viruses, such as herpesviruses (Mackie (2003) Paediatr. Respir. Rev., 4:84-90). Studies of immunocompetent patients have shown that a high percentage (>30%) of patients with RSV or rhinovirus infections showed serological evidence of concomitant infection by another virus. An analysis of mixed viral infections reported that respiratory syncytial virus, influenza viruses, adenoviruses, and parainfluenza viruses were the most common viruses in the mixed respiratory infections, with the combination of RSV and influenza being the most frequent co-infection (Waner (1994) Clin. Microbiol. Rev., 7:143-151). Coinfection by human metapneumovirus and respiratory syncytial virus has also been reported to be common (Greensill et al (2003) Emerg. Infect. Dis., 9:372). Mixed viral-bacterial and viral-fungal respiratory infections are also well documented. See, for example, Korppi et al. (1989) Pediatr. Infect. Dis. J, 8:687-692; Korppi et al. (1990) Scand. J. Infect. Dis., 22:307-312;Korppi et al. (1991) Acta Paediatr. Scand., 80:413-417; Korppi et al. (1993) Eur. J. Pediatr., 152:24; Hament et al. (1999) FEMS Immunol. Med Microbiol., 26:189-195; and Tristram et al. (1988) Arch. Pediatr. Adolesc. Med., 142:834-836, which are incorporated by reference in their entirety herein.

Acute otitis media, though generally thought of as a bacterial infection of the middle ear, can be associated with or influenced by viral pathogens (Heikkinen and Chonmaitree (2003) Clin. Microbiol. Rev., 16:230-241; Canafax et al. (1998) Pediatr. Infect. Dis. J., 17:149-156; and Ruuskanen et al. (1989) Pediatr. Infect. Dis. J., 8:94 which are incorporated by reference in their entirety herein). Similarly, an antecedent infection by certain respiratory viruses, especially influenza A, appears to increase incidence and severity of Streptococcus pneumoniae-caused otitis media (see, for example, Tong et al. (2000) Ann. Otol. Rhinol. Laryngol., 109:1021-1027; Tong et al. (2001) Infect. Immun., 69:602-606; and Tong et al. (2002) Infect. Immun., 70:4292-4301, which are incorporated by reference in their entirety herein).

Meningitis and other central nervous system infections may be viral or bacterial in etiology. Multiple infective agents have been demonstrated (see, for example, Eglin et al. (1984) Lancet, 2(8409):984; Squadrini et al. (1977) Lancet, 1(8026):1371; and Krasinski et al. (1987) Am. J. Epidemiol., 125:499-508, which are incorporated by reference in their entirety herein), with mixed bacterial infections in meningitis having been relatively more commonly reported in the medical literature than mixed viral-bacterial infections (Sferra and Pacini (1988) Pediatr. Infect. Dis. J., 7:552-556, which is incorporated by reference in its entirety herein). Eukaryotic pathogens, such as Giardia lamblia, Entamoeba histolytica, Naegleria fowleri, Cyclospora cayetanensis, Toxoplasma gondi, Cryptosporidium parvum, Acanthomoeba species, microsporidia, and other protozoan pathogens, can also cause central nervous system infections.

Infections of the digestive tract are caused by many pathogens, including viruses, bacteria, and protozoans (Leclerc et al. (2002) Crit. Rev. MicrobioL, 28:371-409, which is incorporated by reference in its entirety herein). Mixed infections, including mixed viral-bacterial infections, have been associated with gastroenteritis (see, for example, Cramblett and Siewers (1965) Pediatrics, 35:885-898; Zavate et al. (1988) Virologie, 39:131-136; Bettelheim et al. (2001) Comp. Immunol. Micorbiol. Infect. Dis., 24:135-142, which are incorporated by reference in their entirety herein).

Viral infections can precede, and overlap with, bacterial infections. In cases of respiratory diseases, it is believed that viral infection predisposes the patient to bacterially caused disease (see, for example, Hament et al. (1999) FEMS Immunol. Med Microbiol., 26:189-195; Korppi (2002) APMIS, 110:515). There is also evidence that a preceding infection by adenovirus and possibly influenza B virus may likewise predispose a subject to bacterial meningitis (Krasinski et al. (1987) Am. J. Epidemiol., 125:499-508). Similarly, viral infection of the respiratory tract often precedes and exacerbates cases of otitis media, and prevention of viral infection by immunization has now been recognized as an important way of preventing acute otitis media (Heikkinen and Chonmaitree (2003) Clin. Microbiol. Rev., 16:230-241). Conversely, coinfection (or superinfection) by a bacterial pathogen may make a viral infection more severe or protracted (see, for example, Jarstrand & Tunevall (1975) Scand. J. Infect. Dis., 7:243-247; Krell et al. (2003) Infection, 5:353-358; Thomas et al. (2003) Pediatr. Infect. Dis. J., 22:201-202, which are incorporated by reference in their entirety herein). The determination of all pathogens, whether viral, bacterial, or eukaryotic (including unfungal and protozoan pathogens), that may be involved in the etiology of a disease condition is therefore essential in allowing prompt treatment with the appropriate antiviral, antibacterial, or other antibiological agents. Rapid and accurate diagnosis is especially important in diseases that have a high fatality rate or serious sequelae, such as occurs in meningitis. Accurate differentiation between viral infections from non-viral infections (especially bacterial infections) is important for avoiding the inappropriate use of antibiotics and the associated adverse effects of antibiotics and risk of developing antibiotic resistance. Diagnosis of viral infections uncomplicated by non-viral infections gives the clinician the option of prescribing antiviral drugs.

### BRIEF SUMMARY OF THE INVENTION

The present invention relates to methods of simultaneously detecting multiple infectious organisms. In particular, the present invention relates to methods of simultaneously determining the presence or absence in a sample of one or more members of a group of pathogens including at least one viral pathogen and at least one non-viral pathogen. The present invention also discloses and claims kits for carrying out the methods of the invention.

One aspect of the present invention includes a method of simultaneously determining the presence or absence in a sample of one or more members of a group of pathogens including at least one viral pathogen and at least one non-viral pathogen, the method including, for each member of the group of pathogens, performing at least one assay for at least one epitope derived from the member, wherein the at least one assay includes: (a) providing at least one binding agent capable of specifically binding to the at least one epitope derived from the member; (b) contacting the at least one binding agent directly to the sample; (c) allowing the at least one binding agent provided to specifically bind to and form a complex with the epitope; and (d) detecting the complex, wherein the detection is positive if the concentration of the member in the sample is greater than or equal to a reference concentration, and the detection is negative if concentration of the member is less than the reference concentration.

Another aspect of the present invention includes assays and kits for carrying out the method of the invention. Such assays and kits may be designed for detection of one or more members of a group of pathogens (including at least one viral pathogen and at least one non-viral pathogen), for example, a group of pathogens whose members can individually or severally cause similar symptoms of disease.

### BRIEF DESCRIPTION OF THE DRAWINGS

FIG. 1 depicts a non-limiting example of a device for performing assays of the present invention, as described in the Examples. This device includes a hinged cardboard housing equipped with a window to allow the viewing of multiple test line results. The device has a recess on one side, containing a preformed plastic well (1) for receiving a swab sample; an overlabel (2); a preassembled test strip (3), prepared as described in Example 1; a viewing window (4); and an optional, lightly adhesive liner (5). The preassembled test strip (3) includes a conjugate pad (6) positioned between conductive pads (7) that direct liquid flow. The conjugate pad contains binding agents that have been temporarily immobilized onto or into the conjugate pad. The test strip also includes a membrane (8) containing the multiple test line reading zone, and an absorbent pad (9). The multiple test line reading zone includes a test line for each of the pathogens detectable by the device.

### DETAILED DESCRIPTION OF THE INVENTION

Introduction

The present invention recognizes the need for, and provides, methods that answer the need for a rapid and accurate determination of the presence or absence of one or more pathogens, for example, determination of whether a subject is infected with a viral pathogen, a non-viral pathogen, or both. The methods can be of use in determining the type of treatment appropriate to an infection, such as whether or not a subject should be treated with antivirals, antibacterials, or other antibiologics. The methods are preferably inexpensive and technically simple, and most preferably do not require expensive or complex instrumentation or equipment. Preferably, such methods do not entail the time and expense of culturing, and most preferably are rapid enough to allow use as a point-of-care diagnostic, such as when used in a clinician's office to provides result within the time of a patient's visit.

As a non-limiting introduction to the breadth of the present invention, the present invention includes several general and useful aspects, including:

1) A method of simultaneously determining the presence or absence in a sample of one or more members of a group of pathogens including at least one viral pathogen and at least one non-viral pathogen, the method including, for each member of the group of pathogens, performing at least one assay for at least one epitope derived from the member, wherein the at least one assay includes: (a) providing at least one binding agent capable of specifically binding to the at least one epitope derived from the member; (b) contacting the at least one binding agent directly to the sample; (c) allowing the at least one binding agent provided to specifically bind to and form a complex with the epitope; and (d) detecting the complex, wherein the detection is positive if the concentration of the member in the sample is greater than or equal to a reference concentration, and the detection is negative if concentration of the member is less than the reference concentration.

2) Assays and kits for carrying out the method of the invention.

Further objectives and advantages of the present invention will become apparent as the description proceeds and when taken in conjunction with the accompanying drawings. To gain a full appreciation of the scope of the present invention, it will be further recognized that various aspects of the present invention can be combined to make desirable embodiments of the invention.

Throughout this application various publications are referenced. The disclosures of these publications are hereby incorporated by reference, in their entirety, in this application. Citations of these documents are not intended as an admission that any of them are pertinent prior art. All statements as to the date or representation as to the contents of these documents is based on the information available to the applicant and does not constitute any admission as to the correctness of the dates or contents of these documents.

Unless defined otherwise, all technical and scientific terms used herein have the same meaning as commonly understood by one of ordinary skill in the art to which this invention belongs. Generally, the nomenclature used herein and the manufacture or laboratory procedures described below are well known and commonly employed in the art. The technical terms used herein have their ordinary meaning in the art that they are used, as exemplified by a variety of technical dictionaries. Where a term is provided in the singular, the inventors also contemplate the plural of that term. The nomenclature used herein and the procedures described below are those well known and commonly employed in the art. Where there are discrepancies in terms and definitions used in references that are incorporated by reference, the terms used in this application shall have the definitions given herein. Other technical terms used herein have their ordinary meaning in the art that they are used, as exemplified by a variety of technical dictionaries (for example, Chambers Dictionary of Science and Technology, Peter M. B. Walker (editor), Chambers Harrap Publishers, Ltd., Edinburgh, UK, 1999, 1325 pp.). The inventors do not intend to be limited to a mechanism or mode of action. Reference thereto is provided for illustrative purposes only.

I. Method for Simultaneously Detecting Viral and Non-Viral Pathogens

The present invention includes a method of simultaneously determining the presence or absence in a sample of one or more members of a group of pathogens including at least one viral pathogen and at least one non-viral pathogen, the method including, for each member of the group of pathogens, performing at least one assay for at least one epitope derived from the member, wherein the at least one assay includes: (a) providing at least one binding agent capable of specifically binding to the at least one epitope derived from the member; (b) contacting the at least one binding agent directly to the sample; (c) allowing the at least one binding agent provided to specifically bind to and form a complex with the epitope; and (d) detecting the complex, wherein the detection is positive if the concentration of the member in the sample is greater than or equal to a reference concentration, and the detection is negative if concentration of the member is less than the reference concentration.

The method of the present invention may be applied to any sample that is suspected of having one or more members of a group of pathogens including at least one viral pathogen and at least one non-viral pathogen. Such samples may include pathology or diagnostic samples, experimental or research samples, or environmental samples. A pathology or diagnostic sample may be from a subject, such as a human subject or a veterinary subject, especially a human or veterinary subject suspected of having a disease caused by an infectious organism. Human subjects may be infants, children, and adult humans of any age. Veterinary subjects may be immature or mature animals of economic, domestic, or research interest (including, but not limited to, dogs, cats, cattle, sheep, goats, swine, rabbits, rats, mice, fish, birds, and non-human primates). Diseases of interest may be any disease that is believed to be caused by at least one pathogen, particularly where the at least one pathogen may be viral or non-viral or a mixture of pathogens. Such diseases include, but are not limited to, respiratory tract infections (such as, but are not limited to, pneumonia, influenza and influenza-like illnesses, sinusitis, bronchitis, tonsillitis, pharyngitis, laryngitis, croup, bronchiolitis, chronic obstructive pulmonary disease), acute otitis media, conjunctivitis, meningitis and other central nervous system infections, and digestive tract infections (such as gastroenteritis or diarrhoea). Where a disease includes infection by multiple pathogens, such infections may be co-infections or superinfections, and may occur concurrently or in succession (with or without overlap of infections).

Where the sample is from a human subject, the sample may include whole cells, tissues, organs, biopsies, biological materials and fluids (for example, blood, serum, plasma, urine, cerebrospinal fluid, synovial fluid, sputum, saliva, semen, tears, and feces), swabs, washes, lavages, discharges, or aspirates (for example, nasal, oral, nasopharyngeal, orophayrngeal, esophagal, gastric, rectal, or vaginal, swabs, washes, ravages, discharges, or aspirates), and extracts or derivatives thereof. For use in the method of the present invention, samples may need minimal preparation (for example, collection into a suitable container), or more extensive preparation (such as, but not limited to, removal, inactivation, or blocking of undesirable material, such as contaminants, undesired cells or cellular material, or endogenous enzymes; treatment with buffers, detergents, surfactants, enzymes, denaturants, reductants, oxidizers, or other reagents; subjection to heat, cold, pressure, vacuum, or other physical treatments; filtration, centrifugation, size selection, or affinity purification; cell fixation, permeabilization, or lysis; and concentration or dilution). Samples may be treated with one or more preparation techniques or one or more preparation reagents. In one embodiment, a sample may use one preparation technique or reagent to enable or facilitate assaying for one or more viral pathogens, and another preparation technique or reagent to enable or facilitate assaying for one or more non-viral pathogens. In a more preferred embodiment, an identical preparation technique (for example, collection of the sample and treatment with the same reagent or reagents) is suitable for treating all members of the group of pathogens of interest. Most preferably, such an identical preparation technique is simple, rapid, and inexpensive.

The method of the present invention can simultaneously determine the presence or absence in a sample of one or more members of a group of pathogens including at least one viral pathogen and at least one non-viral pathogen. By “simultaneously determine” is meant to determine substantially at the same time or substantially within the same narrow time frame. A narrow time frame may be less than about one second, or less than about 1 minute, or less than about 5 minutes, or less than about 15 minutes. Most preferably, the presence or absence in a sample of all of the members of the group of pathogens can be determined within a relatively short period of time, such as during the time of a patient's consultation with a physician or other health care provider, thus permitting the timely prescription of appropriate therapy.

The group of pathogens is generally selected from at least one viral pathogen and at least one non-viral pathogen that each are capable of causing similar or identical symptoms of disease, for example, symptoms of upper respiratory infection, symptoms of meningeal inflammation, or symptoms of gastrointestinal distress. The method can be especially useful in distinguishing viral infections from bacterial or eukaryotic infections, and can additionally be valuable in identifying the particular pathogenic species, type, group, or strain.

Viral pathogens include, but are not limited to, respiratory syncytial virus (RSV) types A and B, influenza A, B, and C viruses, human parainfluenza viruses (hPIV) 1, 2, and 3, human metapneumovirus (hMPV), cytomegaloviruses, adenoviruses (AV), rhinoviruses, coronaviruses, enteroviruses, herpesviruses, enteric adenoviruses, rotavirus groups A, B, and C, astroviruses, sapoviruses, toroviruses, caliciviruses (including noroviruses, Norwalk-like viruses, and Norwalk viruses), herpes viruses, human immunodeficiency viruses, varicella-zoster viruses, polioviruses, arboviruses, mumps viruses, measles viruses, pox viruses, vaccinia viruses, Epstein-Barr viruses, rubella viruses, hantaviruses, echoviruses, coxsackieviruses, and polymyxoviruses. Non-viral pathogens include pathogenic bacteria (including mycoplasmas) and eukaryotic pathogens (including fingi and protozoans). Pathogenic, or potentially pathogenic bacteria of interest include, but are not limited to, (1) bacteria that can be associated with respiratory infections, such as Acinetobacter species, viridans streptococci, beta-hemolytic streptococci (including group A beta-hemolytic streptococci such as Streptococcus pyogenes), non-hemolytic streptococci, Streptococcus pneumoniae, staphylococci (including coagulase-negative staphylococci and Staphylococcus aureus), micrococci, Corynebacterium species (including Corynebacterium diphtheriae), Neisseria species (including Neisseria meningitidis and Neisseria gonorrhoeae), Cryptococcus neoformans, Mycoplasma species (including Mycoplasma pneumoniae), Haemophilus influenzae (both serotypable and non-typable), Haemophilus parainfluenzae, Chlamydia pneumonia, Moraxella (Branhamella) catarrhalis, enterobacteria, Lactobacillus species, Veillonella species, Mycobacterium species, Pseudomonas species, Klebsiella species (including Klebsiella ozaenae or Klebsiella pneumoniae), Bordatella pertussis, Eikenella corrodens, Bacteroides species, Peptostreptococcus species, Actinomyces species, Nocardia species, spirochaetes; (2) bacteria that can be associated with digestive tract infections, such as Bacillus species (including Bacillus cereus), Staphylococcus aureus, Clostridium perftingens, Clostridium difficile, Campylobacter jejuni, Listeria monocytogenes, pathogenic Escherichia coli, E coli O157:H7, Salmonella species, Shigella species, Vibrio species, Yersinia species, Aeromonas species, Pleisiomonas species, and Helicobacter pylori; and (3) bacteria that can be associated with meningitis and other central nervous system infections, such as group B streptococci (including Streptococcus pyogenes), Streptococcus agalactiae, Streptococcus mitis, non-group B streptococci, Streptococcus pneumoniae, Staphylococcus species (including Staphylococcus epidermidis and Staphylococcus aureus), Listeria monocytogenes, Klebsiella pneumoniae, Serratia marcescens, Enterococcus faecium, Enterococcus faecalis, Proteus mirabilis, Mycobacterium tuberculosis, Escherichia coli, Legionella pneumophilia, Corynebacterium species, Haemophilus species (including Haemophilus influenzae), Neisseria species (including Neisseria meningitidis and Neisseria gonorrhoeae), Moraxella lacunata, pseudomonads (including Pseudomonas aeruginosa), Borrelia burgdorferi, and Chlamydia species. Eukaryotic pathogens of interest include, but are not limited to, Histoplasma capsulatum, Coccidioides immitis, Blastomyces dermatitidis, Paracoccidioides brasiliensis, a Candida species, an Aspergillus species, a Mucor species, Cryptococcus neoformans, Pneumocystis carinii, and other fungal pathogens, and Giardia lamblia, Entamoeba histolytica, Naegleriafowleri, Cyclospora cayetanensis, Toxoplasma gondi, Cryptosporidium parvum and other cryptosporidia, Acanthomoeba species, microsporidia, and other protozoan pathogens (see, for example, Marshall et al. (1997) Clin. Microbiol. Rev., 10:67-85, which is incorporated by reference in its entirety herein).

The method includes, for each member of the group of pathogens of interest, performing at least one assay for at least one epitope derived from the member of the group of pathogens. In some embodiments of the method, an assay may be performed for more than one epitope derived from the member, for example, in order to verify with increased certainty the presence or absence of the member, or in order to distinguish between different strains or types of a given member. More than one assay may be performed for a given epitope of interest. Each at least one assay may be performed (1) in parallel with but separately from another assay, (2) sequentially prior to or following another assay, or (3) coincidentally with another assay. Thus, as described below under the heading “II. Assays and kits for simultaneously detecting viral and non-viral pathogens”, the method of the present invention can be performed using kits consisting of a single test device that assays all epitopes of interest, or of multiple test devices, wherein each such single or multiple test devices can make use of single or multiple paths of fluid flow.

The term “epitope” is used here to generally encompass a molecular or multi-molecular structure or site that is specifically recognized and can be bound by a binding agent, such binding generally involving non-covalent interactions. Examples of such generally non-covalent binding of a binding agent to an epitope include, but are not limited to, the binding of an antibody or antibody-like molecule to an antigen, the binding of a T-cell receptor to an antigen, the binding of a receptor to a ligand, the binding of an aptamer to a peptide or other target, the binding of an enzyme to a co-factor or to an enzymatic substrate, and the binding of avidin to biotin. An epitope may represent a much larger antigen or analytical target; for example, an epitope consisting of a small peptide may represent a large protein or proteinaceous complex, or an epitope consisting of a polysaccharide may represent an entire cell. Portions of the molecular structure not directly in contact with the binding agent may affect the binding of an epitope to a binding agent. Binding of the binding agent to the epitope may additionally include covalent bonding, for example, where a disulfide or other covalent bond is formed between the binding agent and the epitope. The at least one epitope derived from the member can be any suitable epitope, including, but not limited to, peptides, polypeptides, proteins, glycoproteins, carbohydrates, lipids, glycolipids, lipoproteins, nucleic acids, antigens, enzymes, receptors, cell wall components, whole cells or intact virions of the member, fragments of the member, substances (such as primary metabolites, secondary metabolites, toxins, enzymes, and exopolymers) produced or secreted by the member, and combinations thereof. Epitopes may include an entire molecule or part of a molecule, or may include all or parts of more than one molecule.

The at least one epitope can optionally be modified, for example, by physical or chemical modification. Modification of the at least one epitope can include any suitable modification, including, but not limited to, treatment with chemical reagents or enzymes, oxidation or reduction, labelling with a detectable or bindable or reactable label, and covalent or non-covalent attachment of the epitope to a separate moiety, molecule, molecular structure, surface, or combination thereof. Non-limiting examples of modification of the at least one epitope include addition of a chemically reactive functional group to the epitope, removal of portions of the epitope (for example, by deglycosylation or by reductive or enzymatic cleavage of covalent bonds), and introduction of a bindable moiety (for example, labelling the epitope with a bindable biotin or other ligand). In some embodiments, the at least one epitope may occur naturally free in solution or suspension, or be made thus, for example, by treatment with appropriate chemical reagents, enzymes, or physical treatments. In other embodiments, the at least one epitope may occur naturally attached to a molecular structure, a molecular complex, a membrane, a cellular component, a whole cell or virion, a porous or solid substrate, or a combination thereof, or be made thus by covalent or non-covalent means or a combination thereof.

Each at least one assay for at least one epitope derived from the member of the group of pathogens includes the step of providing at least one binding agent capable of specifically binding to the at least one epitope derived from the member. Binding agents can be virtually any molecule or combination of molecules capable of recognizing and binding the at least one epitope. Such binding agents can include, without limitation, peptides, polypeptides, antibodies, antibody fragments, fusion proteins, chimeric or hybrid molecules, nucleic acids, nucleic acid mimics (for example, peptide nucleic acids), carbohydrates, cell surface antigens, receptors, ligands, or combinations thereof.

In one preferred embodiment, the at least one binding agent includes an antibody (monoclonal or polyclonal, natural, modified, or recombinant) or an antibody fragment (such as an Fab fragment or single-chain antibody variable region fragment). Antibodies may be natural, modified, or recombinant. Antibody fragments include, but are not limited to, F(ab′)₂ fragments, Fab′ fragments, Fab fragments, Fv fragments, and complementarity determining regions (CDRs). Recombinant antibodies include, but are not limited to, single-chain antibody variable region fragments (scFv), miniantibodies (Müller et al. (1998) FEBS Lett., 432:45-49), antibody fusion proteins, and the like (see, for example, “Antibody Engineering”, R. Kontermann and S. Dübel, editors, Springer-Verlag, Berlin Heidelberg, 790 pp.). Antibodies can be monovalent or polyvalent, such as divalent (Plückthun and Pack (1997) Immunotechnology, 3:83-105; Pack et al. (1995) J. Mol. Biol., 246:28-34). Antibodies can be monospecific or polyspecific, such as bispecific (Müller et al. (1998) FEBS Lett., 432:45-49). Methods of preparing, modifying, and using such antibodies or antibody fragments are known in the art (see, for example, “Antibodies: A Laboratory Manual”, E. Harlow and D. Lane, editors, Cold Spring Harbor Laboratory, 1988, 726 pp; “Monoclonal Antibodies: A Practical Approach”, P. Shepherd and C. Dean, editors, Oxford University Press, 2000, 479 pp.; and “Chicken Egg Yolk Antibodies, Production and Application: IgY-Technology (Springer Lab Manual)”, by R. Schade et al., editors, Springer-Verlag, 2001, 255 pp.). All references cited in this paragraph are incorporated by reference in their entirety herein.

The at least one binding agent can include an antigen, such as an antigen capable of specifically binding to an antibody that recognizes an epitope derived from the member of the group of pathogens. In other embodiments, the binding agent can include a nucleic acid or nucleic acid mimic aptamer that binds a target such as a peptide or small molecule, or a receptor that binds a ligand, or a ligand that binds a receptor. The binding agent may be capable of binding to a mimotope, such as a peptide, that mimics an epitope derived from the member of the group of pathogens (see, for example, Kieber-Emmons (1998) Immunol. Res., 17:95-108; Shin et al. (2001) Infect. Immun., 69:3335-3342; Beenhouwer et al. (2002) J Immunol., 169:6992-6999; Hou and Gu (2003) J. Immunol., 170:4373-4379; and Tang et al. (2003) Clin. Diagn. Lab. Immunol., 10:1078-1084, which are incorporated by reference in their entirety herein).

Where the at least one epitope is optionally modified, for example, by physical or chemical modification, the at least one binding agent can be capable of specifically binding to the modified at least one epitope. In one embodiment, the binding agent may specifically bind to a modification of the at least one epitope, such as to a bindable moiety on the at least one epitope. In another embodiment, the binding agent may bind to the epitope by a non-covalent interaction, and then become affixed to the epitope by a covalent interaction.

The at least one binding agent can optionally include a functional group (such as a chemically reactive moiety or cross-linking moiety) or a detectable label. Detectable labels include, but are not limited to, fluorophores, luminophores, dyes, pigments, members of resonance energy transfer pairs, detectable nuclei (including radioactive isotopes and non-radioactive isotopes), spin labels, lanthanides, magnetic labels, detectable nucleic acids, metals, particles (such as, but not limited to, beads, fibers, or particles made of gold or other metals, magnetic or paramagnetic substances, glass, silicates, ceramics, latex, polymers, or composites), enzymes, antigenically recognizable structures (for example, digoxin or digoxigenin), and bindable moieties (for example, receptors, ligands, polyhistidine tags, biotin, or avidin). Methods to introduce such functional groups or detectable labels are known in the art (see, for example, R. P. Haugland, “Handbook of Fluorescent Probes and Research Products”, 9^{th} edition, J. Gregory (editor), Molecular Probes, Inc., Eugene, Oreg., USA, 2002, 966 pp.; Seitz and Kohler (2001), Chemistry, 7:3911-3925; Pierce Technical Handbook, Pierce Biotechnology, Inc., 1994, Rockford, Ill.; and Pierce 2003-2004 Applications Handbook and Catalog, Pierce Biotechnology, Inc., 2003, Rockford, Ill., which are incorporated by reference in their entirety herein).

The at least one binding agent may be free in solution, or may be temporarily or permanently affixed, directly or indirectly, onto a separate moiety, molecule, molecular structure, or surface. In one non-limiting example of direct immobilization, the at least one binding agent can be temporarily immobilized by drying or otherwise transiently binding onto a surface or into a matrix, wherein addition of a fluid can cause the binding agent to become mobile. Temporary immobilization may include the use of covalent or non-covalent means. In one embodiment, the binding agent can be temporarily immobilized by a covalent bond that can be cleaved, for example, by reduction or enzymatic reaction or by other physical or chemical treatment. In another embodiment, the binding agent may be temporarily immobilized by non-covalent means including, but not limited to, specific interactions (such as between the binding agent and a molecule that recognizes and binds the binding agent), physical adsorption, hydrophobic or hydrophilic interaction, magnetic forces, ionic interactions, electrostatic interactions, van der Waals forces, and combinations thereof. In another non-limiting example of direct immobilization, the binding agent can be permanently immobilized by covalent or non-covalent attachment to a porous or non-porous surface, such as to a bead, fiber, particle, matrix, membrane, microplate well, tube, chip, or slide. In a non-limiting example of indirect immobilization, the at least one binding agent can be affixed by covalent or non-covalent means (such as by passive adsorption or avidin/biotin binding) to particles (for example, beads or fibers or particles of latex, gold or other metals, or magnetic or paramagnetic materials), and the binding agent-bearing particles temporarily or permanently immobilized onto a surface or within a matrix. In another non-limiting example of indirect immobilization, the at least one binding agent can be affixed using a linking moiety, such as a cross-linking molecule or a multivalent molecule (such as avidin), to a separate moiety, molecule, molecular structure, matrix, or surface.

In one embodiment, the at least one binding agent binds monovalently to the epitope of interest. In another embodiment, the binding agent binds multivalently, for example bivalently and optionally bispecifically, to the epitope (or mimotope) of interest, and may bind more than a single epitope together in a multi-epitope (and optionally multi-binding agent) complex. In some embodiments, one unit of the binding agent can bind to one unit of the epitope of interest, and in other embodiments, more than one unit of the binding agent can bind to one unit of the epitope of interest and form a multi-binding agent (and optionally multi-epitope) complex.

The at least one binding agent can be used in more than one form or type, for example, where the binding agent is an antibody or antibody fragment and is used in a sandwich assay that involves an unlabelled binding agent to immobilize the epitope and a detectably labelled binding agent that binds the same epitope. In methods of the invention that use such sandwich assays, any suitable order of the binding steps may be used. Thus, in one non-limiting example, the epitope may be captured and immobilized by a first form of a binding agent, followed by a second form of the binding agent binding to the immobilized epitope to form the detectable complex. In another non-limiting example, a first form of a binding agent binds the epitope to form a detectable complex, followed by immobilization of the complex by capture by a second form of the binding agent.

The at least one binding agent's ability to specifically bind to an epitope derived from the member of the group of pathogens can be improved by means known in the art, for example, by selection of a peptide sequence based on panning methods (see, for example, Coomber (2001) Methods Mol. Biol., 178:133-145; Zhou et al. (2002) Proc. Natl. Acad. Sci. USA, 99:5241-5246; Fehrsen and du Plessis (1999) Immunotechnology, 4:175-184; Deng et al. (1994) J. Biol. Chem., 269:9533-9538; Burioni et al. (1998) Res. Virol., 149:327-330; Boel et al. (1998) Infect. Immun., 66:83-88; and Parsons et al. (1996) Protein Eng., 9:1043-1049, which are incorporated by reference in their entirety herein).

Improvement of the at least one binding agent's ability to bind to an epitope derived from the member of the group of pathogens (or to a mimotope mimicking the epitope) can use display methods as are known in the art, including displaying on a polypeptide (Kamb, et al., U.S. Pat. No. 6,025,485; Christmann et al., 1999, Protein Eng., 12:797; Abedi et al., 1998, Nucleic Acids Res., 26:623; Peelle et al., 2001, J. Protein Chem., 20:507), a phage (He, 1999, J. Immunol. Methods, 231:105; Smith, 1985, Science, 228:1315), a ribosome (Schaffitzel et al., 1999, J. Immunol. Methods, 231:119; Roberts, 1999, Curr. Opin. Chem. Biol., 3:268), an mRNA (Wilson et al., 2001, Proc. Natl. Acad. Sci., 98:3750), or a yeast cell surface (Yeung and Wittrup, 2002, Biotechnol. Prog., 18:212; Shusta et al., 1999, J. Mol. Biol., 292:949), a bacterial cell surface (Leenhouts et al., 1999, Antonie Van Leeuwenhoek, 76:367; Christmann et al., 2001, J. Immunol. Methods, 257:163), or a bacterial spore surface (Wittrup, 2001, Curr. Opin. Biotechnol., 12:395; Boder and Wittrup, 1998, Biotechnol. Prog., 14:55). All references cited in this paragraph are incorporated by reference in their entirety herein.

Each at least one assay for at least one epitope derived from the member of the group of pathogens also includes the step of contacting the at least one binding agent directly to the sample. The binding agent is contacted directly to the sample, which may have undergone prior minimal or more extensive preparation, but which has not been subjected to culturing or to nucleic acid amplification for the member of the group of pathogens.

Each at least one assay for at least one epitope derived from the member of the group of pathogens also includes the step of allowing the at least one binding agent provided to specifically bind to and form a complex with the epitope. The binding of the at least one binding agent to the epitope can be by any suitable means, including, but not limited to, covalent binding, non-covalent binding, antibody-antigen recognition, receptor-ligand binding, aptamer-nucleic acid binding, physical adsorption, electrostatic forces, ionic interactions, hydrogen bonding, hydrophilic-hydrophobic interactions, van der Waals forces, magnetic forces, and combinations thereof. Where more than one binding agent is used (such as, for example, in a sandwich assay employing two antibodies or other binding agents that bind the same epitope, or that bind different epitopes derived from the same pathogen of interest), the multiple binding agents can bind to the epitope by more than one means. Preferably, the binding agent binds to the epitope with sufficient specificity to give minimal or no non-specific or cross-reactive binding between the binding agent and an epitope derived from sources other than the member of interest (such as from cells or tissues of the human subject, or from other infectious or non-infectious species or strains not of interest). The specific binding of the binding agent to the epitope preferably results in a complex of sufficient stability to be detected.

Where the at least one epitope is modified, the modification of the at least one epitope can occur: (1) at any time or times prior to, during, or after the time when the at least one binding agent is contacted with the sample, (2) at any time or time prior to, during, or after the time when the at least one binding agent specifically binds to and forms a complex with the epitope, (3) at any time or times prior to, during, or after the time when the complex is detected, or (4) in any combination of (1), (2), and (3). Modification of the at least one epitope can occur in more than one way and at more than one time. In a non-limiting example, the at least one epitope is modified by covalent or non-covalent attachment to a porous or solid substrate (such as a fibrous support, a nitrocellulose membrane, a siliceous surface, a magnetic bead, or the like) prior to the time when the at least one binding agent (such as an antibody or antibody fragment, optionally modified, for example, by labelling with a detectable label or by permanent or temporary, covalent or non-covalent, attachment to a porous or non-porous surface) is contacted with the sample and specifically binds to and forms a complex with the epitope. In another non-limiting example, the at least one epitope is modified (for example, by introduction of a chemically reactive moiety or by a detectable label) after it is bound to and complexed with the at least one binding agent. In another non-limiting example, the at least one epitope is modified by introduction of a bindable moiety, then bound to and complexed by the at least one binding agent, then modified again by covalent or non-covalent attachment (for example, by means of the introduced bindable moiety) to a porous or solid support.

Each at least one assay for at least one epitope derived from the member of the group of pathogens also includes the step of detecting the complex, wherein the detection is positive if the concentration of the member in the sample is greater than or equal to a reference concentration, and the detection is negative if concentration of the member is less than the reference concentration.

Detection of the complex can be direct, such as by detection of a label on the at least one binding agent. Alternatively, detection of the complex can be indirect, by any suitable means, including, but not limited to, the use of a second binding agent, such as a secondary antibody bearing a detectable label, or such as a detectably-labelled avidin moiety, where the complex includes a bindable biotin. Useful detectable labels include, but are not limited to, fluorophores, luminophores, dyes, pigments, members of resonance energy transfer pairs, detectable nuclei, spin labels, lanthanides, magnetic labels, detectable nucleic acids, metals, particles (such as, but not limited to, beads, fibers, or particles made of gold or other metals, magnetic or paramagnetic substances, glass, silicates, ceramics, latex, polymers, or composites), enzymes, products of enzymatic reactions, antigenically recognizable structures, and bindable moieties (such as avidin, biotin, antigens, antibodies and the like, ligands, and receptors).

In some embodiments, detection of the complex may make use of instrumentation. Instrumentation that may be suitable for use in the method of the invention includes, but is not limited to, spectrophotometric instrumentation (such as instruments capable of ultraviolet, visual, infrared, Raman, luminescent, fluorescent, and phosphorescent light detection), instrumentation for electrochemical detection (such as coulometry, voltametry, potentiometry, and specific ion detection), gravimetric instrumentation, mass spectrometers, electrophoretic equipment, chromatographic equipment, surface plasmon resonance detectors, magnetic resonance detectors, cameras or microscopes (light, electron, atomic force), charge-coupled devices, thermal analysis instrumentation, and combinations thereof. Some embodiments may also use computerized methods to detect or amplify detection of the complex, for example, using computers to integrate a signal over time, to interpolate between known data points, or to increase signal-to-noise ratios. In other embodiments, detection of the complex can be made without the need for instrumentation, for example, by simple visualization by the naked eye without using magnification, physical amplification of the visual signal, or computerized amplification of a digitized signal. Embodiments not needing expensive or complex equipment are preferred for their simplicity and ease of use in clinical settings, particularly when it is impracticable or uneconomical to use embodiments that require specialized technical training.

Detection of the complex is positive if the concentration of the member of the group of pathogens in the sample is greater than or equal to a reference concentration. Conversely, detection of the complex is negative if the concentration of the member of the group of pathogens in the sample is less than the reference concentration. The reference concentration selected for a given member of the group of pathogens depends on several factors, including, but not limited to, the nature of the binding agent and of the epitope derived from the member, the type of sample, and, where the sample is from a subject, the type of subject (for example, an adult or a child). A reference concentration can be any suitable concentration, and can be established by routine testing. Detection can be linear (such as spectrophotometric measurement of product formation by an enzymatic reaction) or non-linear (such as visual detection of a gold label). Detection is optionally at least semi-quantitative, for example, judged to be greater than or equal to, or less than, a reference value. Detection can be optionally quantitative, wherein a positive detection signal can be correlated to a range of concentrations of the member of the group of pathogens.

In one embodiment of the invention, the sample is from a subject suspected of being diseased by at least one of the members of the group of pathogens. Subjects may be human or veterinary. In some cases, a subject may be an asymptomatic “carrier” of a member of the group of pathogens, that is to say, otherwise healthy but colonized, generally at relatively lower concentrations, by the member, where a relatively higher concentration of the member is associated with symptoms of a disease caused by that pathogen. In such a case, a desirable reference concentration is a concentration below which a sample from a subject who either is not colonized by the member in question, or who is colonized by the member but otherwise healthy, gives a negative detection result. This same reference concentration is preferably a concentration at or above which a sample from a subject who is colonized and diseased by the member gives a positive detection result.

Thus, in one embodiment of the invention, a positive detection result indicates that the subject is at least colonized by the member of the group of pathogens, or is colonized and diseased by that pathogen. In one alternative embodiment of the invention, a negative detection result preferably indicates that the subject is not colonized by the member of the group of pathogens to a level associated with a disease caused by that pathogen.

In yet another embodiment of the invention, the sample is from a subject suspected of being diseased by at least one of the members of the group of pathogens, wherein a very low concentration of the at least one member is sufficient to indicate that the subject is likely to be diseased by that at least one member. In such an embodiment, the reference concentration for that at least one member may be very low, optionally approaching or equal to zero.

A desirable reference concentration preferably yields a positive predictive value (that is to say, the probability that the subject with a positive detection result is diseased by the member of the group of pathogens) of at least about 80%, more preferably of at least about 90%, and most preferably of at least about 95%. A desirable reference concentration preferably yields a negative predictive value (that is to say, the probability that the subject with a negative detection result is not diseased by the member of the group of pathogens) of at least about 80%, more preferably of at least about 90%, and most preferably of at least about 95%.

II. Assays and Kits for Simultaneously Detecting Viral and Non-Viral Pathogens

The method of the present invention may be carried out by means of a suitable assay. Non-limiting examples of suitable assays for performing the method include dipstick or test strip assays, flow-through assays, chromatographic assays, affinity separation assays, lateral flow assays, latex agglutination assays, radioimmunometric assays, enzyme-linked immunosorbent assays, fluorescence assays, luminescence assays, dot blot assays, and combinations thereof. Assays can be run in any suitable format, including, but not limited to, test strips, dipsticks, membranes, filters, microtiter plates, tubes, chips, slides, and flow-through chambers. Preferably, the assay is rapid, most preferably sufficiently rapid to produce results within a relatively brief period of time, such as within the time of a subject's consultation with a physician or other health-care provider.

Kits can be designed for convenience in performing the method, according to the assay used. Kits can include, in addition to a means for performing the assay, means for collecting and appropriately treating a sample (such as appropriate collection containers, a swab, a means to aspirate a sample, a means to biopsy a sample, wash solutions or buffers, chemical or enzymatic reagents, filters, centrifuge tubes, and the like). In some embodiments, the means for collecting and appropriately treating a sample may include more than one means for collecting or more than one means of treating a sample. More preferably, a single means for collecting and a single means for treating a sample are sufficient for performing the method. Kits can include materials (such as gloves and other personal safety equipment, biohazard disposal containers, or decontamination materials) that aid in the safe handling of potentially hazardous samples. Kits can include instructions for the use of the kit, for example, instructions in the form of a brochure, leaflet, pamphlet, booklet, or audiovisual materials.

Non-limiting examples of kits of the invention follow:

1. Kit for influenza and influenza-like illnesses (see, for example, Centers for Disease Control (2001) Morbidity Mortality Weekly Report, 50(44):984-986, which is incorporated by reference in its entirety herein), designed to detect one or more members of a group of pathogens including at least one viral pathogen (such as, but not limited to, an influenza virus, a rhinovirus, a respiratory syncytial virus, an adenovirus, a parainfluenza virus, a coronavirus, and a metapneumovirus) and at least one non-viral pathogen (such as, but not limited to, Streptococcus pneumoniae, Chlamydia pneumoniae, and Mycoplasma pneumoniae). Suitable samples for analysis include, but are not limited to, nasopharyngeal, orophayrugeal, or esophagal swabs or washes or discharges and the like, as well as blood, urine, sputum, or saliva, and extracts or derivatives thereof.

2. Kit for pneumonia, designed to detect one or more members of a group of pathogens including at least one viral pathogen (such as, but not limited to, an influenza virus, a rhinovirus, a respiratory syncytial virus, an adenovirus, a parainfluenza virus, a coronavirus, a hantavirus, a cytomegalovirus, and a metapneumovirus) and at least one non-viral pathogen (such as, but not limited to, a group A streptococcus, Streptococcus pyogenes, Streptococcus pneumoniae, Klebsiella pneumoniae, a Staphylococcus species, Haemophilus influenzae, Chlamydia pneumoniae, Mycoplasma pneumoniae, a Pseudomonas species, Histoplasma capsulatum, Coccidioides immitis, Blastomyces dermatitidis, Paracoccidioides brasiliensis, Candida species, Aspergillus species, Mucor species, Cryptococcus neoformans, and Pneumocystis carinii). Suitable samples for analysis include, but are not limited to, nasopharyngeal, orophayrngeal, or esophagal swabs or washes or discharges and the like, as well as blood, urine, sputum, or saliva, and extracts or derivatives thereof.

3. Kit for bronchitis, designed to detect one or more members of a group of pathogens including at least one viral pathogen (such as, but not limited to, an influenza virus, a rhinovirus, a respiratory syncytial virus, an adenovirus, a parainfluenza virus, a coronavirus, a hantavirus, a cytomegalovirus, and a metapneumovirus) and at least one non-viral pathogen (such as, but not limited to, Mycoplasma species such as Mycoplasma pneumoniae, Chlamydia pneumoniae, Bordatella pertussis, Group A Streptococcus, Streptococcus pyogenes, Moraxella catarrhalis, Haemophilus influenzae, Haemophilus parainfluenzae, and Staphylococcus aureus). Suitable samples for analysis include, but are not limited to, nasopharyngeal, orophayrngeal, or esophagal swabs or washes or discharges and the like, as well as blood, urine, sputum, or saliva, and extracts or derivatives thereof.

4. Kit for sinusitis, designed to detect one or more members of a group of pathogens including at least one viral pathogen (such as, but not limited to, an influenza virus, a rhinovirus, a respiratory syncytial virus, an adenovirus, a parainfluenza virus, a coronavirus, a hantavirus, a cytomegalovirus, and a metapneumovirus) and at least one non-viral pathogen (such as, but not limited to, Streptococcus species such as Streptococcus pneumoniae, Haemophilus influenzae, Staphylococcus species such as Staphylococcus aureus, and Neisseria species, or fungal pathogens). Suitable samples for analysis include, but are not limited to, nasopharyngeal, orophaymgeal, or esophagal swabs or washes or discharges and the like, sinus aspirates or biopsies, blood, urine, sputum, or saliva, and extracts or derivatives thereof.

5. Kit for acute otitis media (see, for example, Jones and Wilson (2002), “Otitis Media”, on-line article at http://www.emedicine.com/ped/topic1689.htm, accessed 21 Jan. 2004), designed to detect one or more members of a group of pathogens including at least one viral pathogen (such as, but not limited to, an influenza virus, a rhinovirus, a respiratory syncytial virus, an adenovirus, a parainfluenza virus, a coronavirus, and a metapneumovirus) and at least one non-viral pathogen (such as, but not limited to, non-typable Haemophilus influenzae and Moraxella catarrhalis, Streptococcus pneumoniae). Suitable samples for analysis include, but are not limited to nasopharyngeal, orophaymgeal, or esophagal swabs or washes or discharges and the like, middle ear fluid, discharges, aspirates, or biopsies, blood, urine, sputum, or saliva, and extracts or derivatives thereof.

6. Kit for conjunctivitis (see, for example, Scott and Luu (2001), “Viral Conjunctivitis”, on-line article at www.emedicine.com/oph/topic84.htm, accessed 22 Jan. 2004; and Marlin (2003), “Bacterial Conjunctivitis”, on-line article at www.emedicine.com/OPH/topic88.htm, accessed 22 Jan. 2004), designed to detect one or more members of a group of pathogens including at least one viral pathogen (such as, but not limited to, an adenovirus, a herpes virus, such as a herpes simplex virus, varicella-zoster virus, an enterovirus, a coxsackievirus, a molluscum contagiosum poxvirus, a vaccinia poxvirus, an influenza virus, an Epstein-Barr virus, a paramyxovirus, and a rubella virus) and at least one non-viral pathogen (such as, but not limited to, a group A streptococcus, Streptococcus pyogenes, Streptococcus mitis, Streptococcus pneumoniae, Staphylococcus epidermidis, Staphylococcus aureus, a Corynebacterium species, Haemophilus species, Haemophilus influenzae, Neisseria species such as Neisseria meningitidis and Neisseria gonorrhoeae, Moraxella lacunata, pseudomonads such as Pseudomonas aeruginosa, and Chlamydia species). Suitable samples for analysis include, but are not limited to, nasopharyngeal, orophayrngeal, or esophagal swabs, washes, or discharges and the like, and blood, urine, sputum, tears, eye discharges, or saliva, and extracts or derivatives thereof.

7. Kit for central nervous system infections, such as meningitis (see, for example, Incesu and Khosla (2003), “Bacterial Meningitis”, on-line article at www.emedicine.com/radio/topic441.htm, accessed 22 Jan. 2004; and Vokshoor and Moore (2004), “Viral Meningitis”, on-line article at: www.emedicine.com/neuro/topic607.htm, accessed 22 Jan. 2004) designed to detect one or more members of a group of pathogens including at least one viral pathogen (such as, but not limited to, a herpes virus, a varicella-zoster virus, an enterovirus, a poliovirus, an arbovirus, a mumps virus, a measles virus, an echovirus, a coxsackievirus, a human immunodeficiency virus, and adenovirus) and at least one non-viral pathogen (such as, but not limited to, Haemophilus influenzae type b, Streptococcus pneumoniae, Neisseria meningitidis, group B streptococci, Streptococcus agalactiae, Streptococcus pyogenes, non-group B streptococci, Staphylococcus species, Listeria monocytogenes, Klebsiella pneumoniae, Serratia marcescens, Pseudomonas aeruginosa, Enterococcusfaecium, Enterococcusfaecalis, Proteus mirabilis, Escherichia coli, Mycobacterium tuberculosis, Legionella pneumophilia, Borrelia burgdorferi). Suitable samples for analysis include, but are not limited to nasopharyngeal, orophayrngeal, or esophagal swabs or washes or discharges and the like, as well as blood, urine, cerebrospinal fluid, tears, sputum, or saliva, and extracts or derivatives thereof.

8. Kit for gastroenteritis (see, for example, Goodgame (2003) “Viral Gastroenteritis”, on-line article at www.emedicine.com/med/topic856.htm, accessed 22 Jan. 2004; and Frye et al. (2002) “Bacterial Gastroenteritis”, on-line article at www.emedicine.com/MED/topic855.htm, accessed 22 Jan. 2004), designed to detect one or more members of a group of pathogens including at least one viral pathogen (such as, but not limited to, a rotavirus, a calicivirus, such as a norovirus, a Norwalk-like virus, a Norwalk virus, and a sapovirus, an astrovirus, and an adenovirus) and at least one non-viral pathogen (such as, but not limited to, Escherichia coli, E. coli O157:H7, Bacillus species, Staphylococcus aureus, Shigella species, Salmonella species, Campylobacter species, Clostridium difficile, Clostridium perftingens, Vibrio species, Listeria monocytogenes, Aeromonas species, Yersinia species, Plesiomonas species, Giardia lamblia, Entamoeba histolytica, Toxoplasma gondii, and cryptosporidia). Suitable samples for analysis include, but are not limited to nasopharyngeal, orophayrngeal, esophagal, gastric, or rectal swabs or washes or discharges and the like, as well as blood, urine, sputum, saliva, feces, and extracts or derivatives thereof.

### EXAMPLES

### Example 1

### Preparation of a Test Strip

This example describes the preparation of a test strip for use in a device for determining the presence or absence in a sample of one or more members of a group of pathogens including at least one viral pathogen and at least one non-viral pathogen.

The test strip includes a conjugate pad, such as, but not limited to, a pad made of glass fiber material or non-woven polyester (for example, Hollingworth & Vose 7304, Web Converting, Holliston, Mass., USA), positioned between conductive pads, such as, but not limited to, pads made of cellulosic paper (for example, Ahlstrom 1281 Paper, 90% cellulose fiber, 10% rayon with traces of polyacrylamide, Web Converting, Holliston, Mass., USA) that direct liquid flow. The conjugate pad contains binding agents, such as detectably labelled antibodies, that have been temporarily immobilized by drying onto the conjugate pad. The test strip also includes a nitrocellulose membrane (for example, Millipore STHF nitrocellulose, catalogue number SA3J727H5, Millipore, Inc., Bedford, Mass., USA) or other suitable membrane (such as, but not limited to, a membrane made of nylon or polyethylene sulfone) containing the multiple test line reading zone. The multiple test line reading zone includes a test line for each of the pathogens detectable by the device. Each test line contains an antibody specific for the particular pathogen, permanently immobilized by adsorption in a stripe. The test strip also includes an absorbent pad that serves as a reservoir for liquid and promotes capillary flow of the sample through the conjugate pad and thence through the nitrocellulose membrane. In this embodiment, the immunochromatographic analysis of the pathogens of interest occurs in and along a single path of fluid flow, wherein the fluid flows through each of the test lines. Alternative embodiments of this device may be designed, such as a device wherein the above described immunochromatographic analysis of individual pathogens is carried out using discrete paths of fluid flow (each containing one or more test lines or the like), such as, but not limited to, in multiple discrete test strips, or in a test strip wherein fluid flow is compartmentalized by any suitable method of separating multiple paths of fluid flow (for example, by treatment with a hydrophobic reagent that prevents aqueous fluids from flowing across the area treated).

Antibodies are selected as appropriate to the antigens or pathogens of interest. Affinity purification is carried out as necessary, by methods well known in the art (see, for example, P. Tijssen, “Practice and theory of enzyme immunoassays”, pp. 173-189, in “Laboratory techniques in biochemistry and molecular biology”, R. Burden and P. VanKnippenberg (editors), Elsevier, Amsterdam, The Netherlands, 1985; “Antibodies: A Laboratory Manual”, E. Harlow and D. Lane, editors, Cold Spring Harbor Laboratory, 1988, 726 pp; “Monoclonal Antibodies: A Practical Approach”, P. Shepherd and C. Dean, editors, Oxford University Press, 2000, 479 pp.; and “Chicken Egg Yolk Antibodies, Production and Application: IgY-Technology (Springer Lab Manual)”, by R. Schade et al., editors, Springer-Verlag, 2001, 255 pp., which are incorporated by reference in their entirety herein).

Gold particles are conjugated to the selected antibodies. Any method for conjugation may be used, a non-limiting example of which is that described by DeMay in “The Preparation and Use of Gold Probes” (in “Immunocytochemistry: Modern Methods and Applications”, 2nd edition, J. M. Polak and S. Van Noorden (editors), Wright, Bristol, England, 1986, 703 pp). In other embodiments, detectable labels other than gold may be used (such as, but not limited to, visible dyes, pigments, magnetic particles or other particulates, enzymes, radioactive isotopes, spin labels, or nucleic acids). The labelled antibodies are mixed with a drying agent (aqueous 5 millimolar sodium tetraborate, pH 8.0, containing 1.0% bovine serum albumin, 0.1% Triton X-100, 2.0% Tween 20, 6.0% sucrose, and 0.02% sodium azide) and temporarily immobilized onto a suitable inert absorbent material (referred to as the “conjugate pad”), such as a non-woven polyester pad able to hold the dried labelled antibodies and to release them when wetted. The conjugate pad is heated sufficiently (for example, to 60 degrees Celsius for 20 minutes) to remove substantially all the liquid, leaving the labelled antibodies temporarily immobilized on the conjugate pad.

The nitrocellulose membrane is prepared by immobilizing the appropriate capture antibodies in individual stripes (referred to as “capture lines” or “test lines”) within the area of the multiple test line reading zone. The capture antibodies, dissolved in a saline solution (0.01 molar phosphate buffered saline, pH 7.4, 1% sucrose, and 0.02% intrawhite dye, 0.05% sodium azide), are applied in stripes. The nitrocellulose is heated sufficiently (for example, to 50 degrees Celsius for 2 minutes) to remove substantially all the liquid, leaving the capture antibodies immobilized on the nitrocellulose membrane, which is then preferably stored desiccated at about 15 to about 30 degrees Celsius to promote permanent absorption of the antibodies to the nitrocellulose.

The absorbent pad that serves as a liquid reservoir and promotes capillary flow can be made of any suitable material. A non-limiting example of suitable material is cellulosic material sold commercially as Ahlstrom 939 (Ahlstrom, Mount Holly Spring, Pa., USA).

The conductive pads, conjugate pad, nitrocellulose membrane, and absorbent pad are assembled together into a test strip, in a manner that can permit fluid flow, for example, by assembly on an adhesive strip. Such an assembled test strip can be further incorporated into assay devices, such as those described in Example 2.

### Example 2

### Immunochromatographic Assay

This example describes a non-limiting embodiment of a method for determining the presence or absence in a sample of one or more members of a group of pathogens including at least one viral pathogen and at least one non-viral pathogen.

In this example, an immunochromatographic assay makes use of the method of the invention to detect the presence or absence of a viral pathogen (influenza A) and two non-viral pathogens (the pathogenic bacteria Streptococcus pneumoniae and Legionella pneumoniae). Such an assay would be desirable, for example, for determining whether a subject suspected of having pneumonia is diseased by any of these pathogens, and to establishing suitable therapy (such as the use or non-use of antibiotics). Similar assays can be designed to detect the presence or absence of additional, or different, pathogens. In one non-limiting example, an assay for meningitis may be designed to detect the presence or absence in a cerebrospinal fluid or nasopharyngeal sample of enteroviruses and the non-viral pathogens Streptococcus pneumoniae, Neisseria meningitidis, and Haemophilus influenzae. In another non-limiting example, an assay for gastroenteritis may be designed to detect the presence or absence in a stool sample of enteroviruses and caliciviruses, and the non-viral pathogens Campylobacter species, Salmonella species, Shigella species, Escherichia coli, and Giardia lamblia.

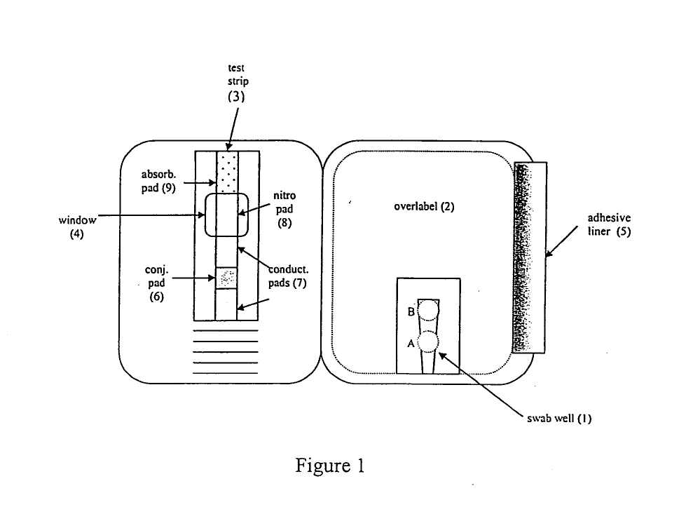

The assay is performed using a lateral flow, immunochromatographic device, as described in U.S. Pat. No. 5,877,028 to Chandler et al., “Immunochromatographic assay device”, issued 2 Mar. 1999, and in U.S. patent applications Ser. No. 09/156,486 and Ser. No. 09/518,165, which are incorporated by reference in their entirety herein. This device comprises a hinged cardboard housing equipped with a window to allow the viewing of multiple test line results (FIG. 1).

The device has a recess on one side, containing a preformed plastic well (1) for receiving a swab sample. An overlabel (2) equipped with two openings is placed over this side of the device with the openings positioned over the well (1) to allow insertion of the swab through the bottom opening (A), so that when the swab is pushed forward, its tip is exposed through the top opening (B). The arrangement of the overlabel, its openings, and the well, combine to hold the swab in the desired position during the assay and to promote the expulsion of sample liquid from the swab.

On the side opposite to that with the well (1), a preassembled test strip, prepared as described in Example 1, (3) is adhered in a position that allows viewing of the multiple test line reading zone through the window (4) when the device is closed. Optionally, the device includes a means to keep the device closed, such as a lightly adhesive liner (5). The preassembled test strip (3) includes a conjugate pad (6) positioned between conductive pads (7) that direct liquid flow. See FIG. 1. The conjugate pad contains binding agents that have been temporarily immobilized by drying onto the conjugate pad. In this non-limiting example, the binding agents include mouse anti-influenza A monoclonal antibodies and affinity purified rabbit anti-Legionella pneumoniae, and rabbit anti-Streptococcus pneumoniae polyclonal antibodies that have been conjugated to a detectable label (gold particles). The test strip also includes a nitrocellulose membrane (8) containing the multiple test line reading zone, and an absorbent pad (9). The multiple test line reading zone includes a test line for each of the pathogens detectable by the device. Each test line contains an antibody specific for the particular pathogen (affinity purified mouse anti-influenza A, rabbit anti-Legionella pneumoniae, and rabbit anti-Streptococcus pneumoniae antibodies) immobilized by adsorption in a stripe.

The assay is carried out with the assembled hinged device. A fibrous Dacron swab is inserted through the opening A into the well (1), resulting in the swab tip being exposed through the opening B. Liquid sample (100 microliters) is pipetted through the opening A to wet the swab. Three drops of “Reagent A” (0.05 molar citrate, 0.25 molar phosphate, pH 7.0, containing 1.4% Tween 20, 0.5% sodium dodecyl sulphate, and 0.05% sodium azide) are added to the swab sample through opening A. The hinged device is closed, and kept closed optionally by the adhesive strip. When the device is closed, the sample liquid contacts the bottom conductive pad of the test strip (3), initiating assay flow. The sample liquid then contacts the conjugate pad and solubilizes the gold-labelled antibodies, allowing their movement onto the nitrocellulose membrane, with excess fluid flowing into the absorbent pad. Antigen present in the sample is bound by the gold-labelled antibodies to form a complex, which is bound by the immobilized antibodies in the test lines. A visually detected signal (a pink-to-purple colored line) is observed when sufficient complex is formed. After sufficient time (such as 15 minutes) has passed, the multiple test line reading zone is viewed through the window of the device, and the results noted as shown in Table 1.

TABLE 1

Test Line

| | Streptococcus | Legionella | |
|---|---|---|---|
| Sample | pneumoniae | pneumoniae | Influenza A |
| Negative urine | Negative | Negative | Negative |
| Streptococcus pneumoniae urine | Positive | Negative | Negative |
| Legionella pneumoniae urine | Negative | Positive | Negative |
| Influenza A in saline | Negative | Negative | Positive |
| Influenza A + Streptococcus pneumoniae urine | Positive | Negative | Positive |
| Influenza A + Legionella pneumoniae urine | Negative | Positive | Positive |

### Example 3

### Immunochromatographic Assay

This example describes a non-limiting embodiment of a method for determining the presence or absence in a sample of one or more members of a group of pathogens including at least one viral pathogen and at least one non-viral pathogen. In particular, this example describes a non-limiting embodiment of a method to simultaneously determine the presence or absence in a sample of viruses and a bacterium that are capable of causing similar disease symptoms in human subjects.

A lateral flow device, similar to devices described, for example, in U.S. Pat. No. 5,877,028, U.S. patent application Ser. Number 09/156,486, and U.S. patent application Ser. No. 09/518,165, and in U.S. patent application Ser. No. 09/397,110, was constructed for the determination of the presence or absence in a sample of influenza A virus, influenza B virus, and Streptococcus pneumoniae. Affinity-purified polyclonal antibodies specific to Streptococcus pneumoniae and two monoclonal antibodies specific respectively to influenza A and B viruses were immobilized in separate capture lines on nitrocellulose. Gold-labelled affinity-purified antibodies specific to each member of this group of pathogens were prepared by covalently coupling colloidal gold particles to the appropriate antibody, and the labelled antibodies temporarily immobilized on a conjugate pad as described above in Example 1.

Samples can consist of nasal, oral, oropharyngeal, or nasopharyngeal swabs, aspirates, washes, lavages, or discharges. In alternative embodiments, other materials (including, but not limited to, urine, blood, serum, plasma, sputum, tissue biopsies, cell extracts, environmental samples, and derivatives thereof) can also serve as suitable samples.

In one specific embodiment, the disease condition of interest that can potentially involve infection by at least member of the group consisting of influenza A virus, influenza B virus, and Streptococcus pneumoniae is acute otitis media. Acute otitis media can be caused by viral pathogens (including respiratory syncytial virus, influenza viruses, parainfluenza viruses, rhinoviruses, and adenoviruses) or by bacterial pathogens (including Streptococcus pneumoniae, Haemophilus influenzae, Staphylococcus aureus, and Moraxella catarrhalis, group A streptococci, Streptococcus pyogenes, Pseudomonas aeruginosa). Otitis media is often associated with respiratory viral infections, and some cases of otitis media may involve both viral and bacterial infections sequentially or concurrently (see, for example, Ruuskanen et al. (1989) Pediatr. Infect. Dis. J., 8:94;.Andrade et al. (1998) Pediatrics, 101:617; and Jones and Wilson (2002), “Otitis Media”, on-line article at http://www.emedicine.com/ped/topic1689.htm, accessed 21 Jan. 2004, which are incorporated by reference in their entirety herein).

The assays for the different pathogens are run using one or more test strips similar to that described in Examples 1 and 2. In one embodiment, each pathogen is assayed for on a separate test strip. In an alternative embodiment, more than one pathogen is assayed for on a single test strip. In various embodiments, for example, influenza A and influenza B viruses may be assayed for on one test strip, and Streptococcus pneumoniae on a second test strip, or each pathogen may be assayed for on separate test strips, or all three pathogens may be assayed for using a single test strip using a single fluid path or more than one fluid path.

When testing for the etiologic agent responsible for otitis media, a suitable sample can be a nasal or nasopharyngeal swab inserted into the nares of a human subject. The swab is removed from the patient's nares, and contacted with a solution, such as a transport medium, to elute the sample. The sample is contacted to the test strip or strips, for example, by insertion of the swab into a device similar to that described in Example 2, closing the device to permit transfer of sample liquid to the bottom conductive pad (or pads) of the test strip (or test strips), thus initiating assay flow. The sample liquid then contacts the conjugate pad (or pads) and solubilizes the gold-labelled antibodies, allowing their movement onto the nitrocellulose membrane (or membranes), with excess fluid flowing into the absorbent pad (or pads). Antigen present in the sample is bound by the gold-labelled antibodies to form a complex, which is bound by the immobilized antibodies in the test lines. A visually detected signal (a pink-to-purple colored line) is observed when sufficient complex is formed. After sufficient time (such as 15 minutes) has passed, the test lines are viewed, and the results noted.

All headings are for the convenience of the reader and should not be used to limit the meaning of the text that follows the heading, unless so specified. Various changes and departures may be made to the present invention without departing from the spirit and scope thereof. Accordingly, it is not intended that the invention be limited to that specifically described in the specification or as illustrated in the drawings, but only as set forth in the claims.

1. A method of simultaneously determining the presence or absence in a sample of one or more members of a group of pathogens including at least one viral pathogen and at least one non-viral pathogen, said method comprising:
for each member of said group of pathogens, performing at least one assay for at least one epitope derived from said member,
wherein said at least one assay comprises:
a) providing at least one binding agent capable of specifically binding to said at least one epitope derived from said member;
b) contacting said at least one binding agent directly to said sample;
c) allowing said at least one binding agent provided to specifically bind to and form a complex with said epitope; and
d) detecting said complex, wherein said detection is positive if the concentration of said member in said sample is greater than or equal to a reference concentration, and said detection is negative if concentration of said member is less than said reference concentration.

2. The method of claim 1, wherein said sample is from a subject suspected of being diseased by at least one of said members.

3. The method of claim 2, wherein said positive detection is optionally quantitative.

4. The method of claim 2, wherein positive detection indicates that said subject is diseased by the member for which said positive detection was made.

5. The method of claim 2, wherein negative detection indicates that said subject is not diseased by the member for which said negative detection was made.

6. The method of claim 2, wherein said disease comprises a respiratory tract infection.

7. The method of claim 6, wherein said respiratory tract infection comprises pneumonia.

8. The method of claim 6, wherein said respiratory tract infection comprises influenza or an influenza-like illness.

9. The method of claim 6, wherein said respiratory tract infection comprises sinusitis.

10. The method of claim 6, wherein said respiratory tract infection comprises bronchitis.

11. The method of claim 6, wherein said respiratory tract infection comprises tonsillitis.

12. The method of claim 6, wherein said respiratory tract infection comprises pharyngitis.

13. The method of claim 6, wherein said respiratory tract infection comprises croup.

14. The method of claim 6, wherein said respiratory tract infection comprises bronchiolitis.

15. The method of claim 6, wherein said respiratory tract infection comprises chronic obstructive pulmonary disease.

16. The method of claim 2, wherein said respiratory tract infection comprises acute otitis media.

17. The method of claim 2, wherein said disease comprises conjunctivitis.

18. The method of claim 2, wherein said disease comprises meningitis.

19. The method of claim 2, wherein said disease comprises a central nervous system infection.

20. The method of claim 2, wherein said disease comprises a gastrointestinal infection.

21. The method of claim 2, wherein said disease comprises a bacterial superinfection.

22. The method of claim 2, wherein said disease comprises a viral superinfection.

23. The method of claim 1, wherein said at least one viral pathogen comprises at least one virus selected from the group consisting of rhinovirus, influenza virus, human metapneumovirus, respiratory syncytial virus, adenovirus, parainfluenza virus, coronavirus, hantavirus, cytomegalovirus, coxsackie virus, herpes simplex virus, and echovirus.

24. The method of claim 1, wherein said at least one non-viral pathogen comprises at least one bacterial pathogen.

25. The method of claim 1, wherein said at least one non-viral pathogen comprises at least one eukaryotic pathogen.

26. The method of claim 1, wherein said at least one non-viral pathogen comprises at least one bacterial pathogen and at least one eukaryotic pathogen.

27. The method of claim 1, wherein said at least one viral pathogen is at least one selected from the group consisting of influenza virus A, influenza virus B, influenza virus C, human metapneumovirus, respiratory syncytial virus A, respiratory syncytial virus B, human parainfluenza virus type 1, human parainfluenza virus type 2, human parainfluenza virus type 3, rhinoviruses, coronaviruses, adenoviruses, hantaviruses, and cytomegaloviruses, and wherein said at least one non-viral pathogen is at least one selected from the group consisting of Streptococcus pneumoniae, Chlamydia pneumoniae, Moraxella catarrhalis, Haemophilus influenzae, Haemophilus parainfluenzae, a Mycoplasma species, Mycoplasma pneumoniae, a group A streptococcus, Streptococcus pyogenes, Klebsiella pneumoniae, a Pseudomonas species, a Staphylococcus species, Staphylococcus aureus, Bordatella pertussis, a Neisseria species, Histoplasma capsulatum, Coccidioides immitis, Blastomyces dermatitidis, Paracoccidioides brasiliensis, a Candida species, an Aspergillus species, a Mucor species, Cryptococcus neoformans, and Pneumocystis carinii.

28. The method of claim 1, wherein said at least one viral pathogen comprises at least one of influenza virus, respiratory syncytial virus, human metapneumovirus, parainfluenza virus, rhinovirus, and adenovirus, and said at least one non-viral pathogen comprises at least one of Streptococcus pneumoniae, Staphylococcus aureus, Haemophilus influenzae, Moraxella catarrhalis, Chlamydia pneumoniae, Haemophilus parainfluenzae, a Neisseria species, and a group A streptococcus.

29. The method of claim 1, wherein said at least one viral pathogen is at least one selected from the group consisting of adenoviruses, herpes viruses, varicella-zoster viruses, enteroviruses, coxsackieviruses, molluscum contagiosum poxviruses, vaccinia poxviruses, influenza viruses, Epstein-Barr viruses, paramyxoviruses, and rubella viruses, and wherein said at least one non-viral pathogen is at least one selected from the group consisting of a group A streptococcus, Streptococcus pyogenes, Streptococcus mitis, Streptococcus pneumoniae, Staphylococcus epidermidis, Staphylococcus aureus, a Corynebacterium species, Haemophilus species, Haemophilus influenzae, Neisseria species such as Neisseria meningitidis and Neisseria gonorrhoeae, Moraxella lacunata, pseudomonads such as Pseudomonas aeruginosa, and Chlamydia species.

30. The method of claim 1, wherein said at least one viral pathogen is at least one selected from the group consisting of herpes viruses, varicella-zoster viruses, enteroviruses, polioviruses, arboviruses, mumps viruses, measles viruses, echoviruses, coxsackieviruses, human immunodeficiency viruses, and adenoviruses, and wherein said at least one non-viral pathogen is at least one selected from the group consisting of Haemophilus influenzae type b, Streptococcus pneumoniae, Neisseria meningitidis, group B streptococci, Streptococcus agalactiae, Streptococcus pyogenes, non-group B streptococci, Staphylococcus species, Listeria monocytogenes, Klebsiella pneumoniae, Serratia marcescens, Pseudomonas aeruginosa, Enterococcus faecium, Enterococcus faecalis, Proteus mirabilis, Escherichia coli, Mycobacterium tuberculosis, Legionella pneumophilia, and Borrelia burgdorferi.

31. The method of claim 1, wherein said at least one viral pathogen is at least one selected from the group consisting of enteroviruses, echoviruses, rotaviruses, caliciviruses, noroviruses, Norwalk-like viruses, Norwalk viruses, sapoviruses, astroviruses, and adenoviruses, and wherein said at least one non-viral pathogen is at least one selected from the group consisting of Escherichia coli, E. coli O157:H7, Bacillus species, Staphylococcus aureus, Shigella species, Salmonella species, Campylobacter species, Clostridium difficile, Clostridium perfringens, Vibrio species, Listeria monocytogenes, Aeromonas species, Yersinia species, Plesiomonas species, Giardia lamblia, Entamoeba histolytica, Toxoplasma gondii, and cryptosporidia.

32. The method of claim 1, wherein said at least one epitope is modified.

33. The method of claim 1, wherein said at least one binding agent comprises an antibody or antibody fragment.

34. The method of claim 1, wherein said at least one binding agent comprises a functional group or a detectable label.

35. The method of claim 1, wherein said at least one binding agent is further capable of binding to a mimotope that mimics said at least one epitope derived from said member.

36. The method of claim 1, wherein said at least one binding agent is used in more than one form.

37. The method of claim 36, wherein said assays comprise at least one sandwich immunoassay.

38. The method of claim 1, wherein said assays comprise at least one immunochromatographic assay.

39. The method of claim 38, wherein said at least one immunochromatographic assay comprises at least one sandwich assay.

40. The method of claim 1, wherein said assays are run in parallel.

41. The method of claim 1, wherein said assays are run sequentially.

42. The method of claim 1, wherein said assays are run coincidently.

43. The method of claim 1, wherein said assays are run in a single path of fluid flow.

44. The method of claim 1, wherein said assays are run in multiple paths of fluid flow.

45. A kit for performing the method of claim 1.

46. The kit of claim 45, comprising at least one immunochromatographic device.

47. The kit of claim 45, wherein said assays are run in a single path of fluid flow.

48. The kit of claim 45, wherein said assays are run in multiple paths of fluid flow.

49. A kit for performing the method of claim 27.

50. The kit of claim 49, comprising at least one immunochromatographic device.

51. The kit of claim 49, wherein said assays are run in a single path of fluid flow.

52. The kit of claim 49, wherein said assays are run in multiple paths of fluid flow.

53. A kit for performing the method of claim 28.

54. The kit of claim 53, comprising at least one immunochromatographic device.

55. The kit of claim 53, wherein said assays are run in a single path of fluid flow.

56. The kit of claim 53, wherein said assays are run in multiple paths of fluid flow.

57. A kit for performing the method of claim 29.

58. The kit of claim 57, comprising at least one immunochromatographic device.

59. The kit of claim 57, wherein said assays are run in a single path of fluid flow.

60. The kit of claim 57, wherein said assays are run in multiple paths of fluid flow.

61. A kit for performing the method of claim 30.

62. The kit of claim 61, comprising at least one immunochromatographic device.

63. The kit of claim 61, wherein said assays are run in a single path of fluid flow.

64. The kit of claim 61, wherein said assays are run in multiple paths of fluid flow.

65. A kit for performing the method of claim 31.

66. The kit of claim 65, comprising at least one immunochromatographic device.

67. The kit of claim 65, wherein said assays are run in a single path of fluid flow.

68. The kit of claim 65, wherein said assays are run in multiple paths of fluid flow.
